(19) United States
(12) Patent Application Publication
(10) Pub. No.: US 2024/0000998 A1
(43) Pub. Date: Jan. 4, 2024

(54) Tissue Customized Platelet-Rich Plasma for Optimized Skeletal Muscle, Cartilage, and Bone Healing

(71) Applicant: University of Pittsburgh - Of the Commonwealth System of Higher Education, Pittsburgh, PA (US)

(72) Inventor: Hongshuai Li, Coralville, IA (US)

(21) Appl. No.: 18/035,138

(22) PCT Filed: Nov. 4, 2021
(86) PCT No.: PCT/US21/58089

§ 371 Date: May 3, 2023

Related U.S. Application Data
(60) Provisional application No. 63/109,482, filed on Nov. 4, 2020.

(51) Int. Cl. A61L 27/36

(52) U.S. Cl. CPC A61L 27/3616; A61L 27/3687; A61L 2430/30; A61L 2430/06; A61L 2430/02

(57) ABSTRACT

Provided herein is a platelet-rich plasma (PRP) product comprising activated PRP releasate in which a deleterious factor that inhibits growth of a target tissue type is removed. The PRP product is useful in growth or regeneration of tissue of the target tissue type, such as muscle, cartilage, or bone. Also provided herein is a method of making a tissue-customized PRP product for growth or repair of a target tissue, such as muscle, cartilage, or bone, comprising removing a deleterious factor for the growth of the target tissue from activated PRP releasate.

### CROSS REFERENCE TO RELATED APPLICATIONS

This application claims the benefit of U.S. Provisional Patent Application No. 63/109,482 filed Nov. 4, 2020, the disclosure of which is incorporated herein by reference in its entirety.

Platelet-rich plasma (PRP) is an emerging biological tool for musculoskeletal tissue healing. It has a significant advantage over other potential therapies since it contains abundant autologous growth factors and is easy to obtain; however, clinical outcomes after PRP treatment are still conflicting and the mechanism of action is largely unclear. In addition, the variations in PRP itself, such as the differences between commercial systems with respect to the preparation protocol, number of platelets, use of anticoagulants, inclusion of leukocytes, and use of activators, represent variables that may influence the biological effects of PRP.

### SUMMARY

Provided herein is a method of preparing a platelet-rich-plasma-derived (PRP-derived) composition for use in regeneration of a target tissue, comprising: removing from activated PRP releasate a deleterious factor of the PRP comprising a bioactive component that inhibits growth, repair, or healing of the target tissue (e.g., such that as compared to PRP from which the PRP-derived composition is made, the PRP-derived composition better promotes growth, repair, or healing of the tissue. Further, a bioactive component that inhibits growth repair, or healing of target tissue may cause abnormal healing, such as scarring, fibrosis, or other effects, that are not found in heathy, normal tissue of the tissue type of the target tissue).

Also provided herein is a method of growth, repair, or healing of a target tissue, in a patient, comprising contacting the target tissue with a platelet-rich-plasma-derived (PRP-derived) composition that is prepared by removal from activated PRP releasate a deleterious factor of the PRP comprising a bioactive component that inhibits growth, repair, or healing of the target tissue, such that as compared to PRP, the PRP-derived composition better promotes growth, repair, or healing of the tissue.

Also provided herein is a platelet-rich-plasma-derived (PRP-derived) composition that is prepared by removal from activated PRP releasate a deleterious factor of the PRP comprising a bioactive component that inhibits growth, repair, or healing of a target tissue.

Further provided herein is a kit comprising a container and, in the container, the PRP-derived composition as described herein.

Also provided herein is a kit comprising: a blood collection tube comprising an anticoagulant; a PRP activation reagent in a vessel; and an affinity-purification reagent comprising a binding reagent specific to, and for binding, a deleterious factor of activated PRP comprising a bioactive component that inhibits growth, repair, or healing of a target tissue, wherein the bioactive component optionally is VEGF, PDGF, TGF-β1, both VEGF and PDGF, both PDGF and TGF-β-1, or noggin.

The following numbered clauses describe various aspects and/or embodiments of the present invention.

Clause 1. A method of preparing a platelet-rich-plasma-derived (PRP-derived) composition for use in regeneration of a target tissue, comprising: removing from activated PRP releasate a deleterious factor of the PRP comprising a bioactive component that inhibits growth, repair, or healing of the target tissue.

Clause 2. The method of clause 1, wherein the bioactive component that inhibits growth, repair, or healing of the target tissue is a cytokine or growth factor.

Clause 3. The method of clause 1, wherein the bioactive component that inhibits growth, repair, or healing of the target tissue has an immunomodulatory effect.

Clause 4. The method of clause 1, wherein the target tissue is cartilage, and wherein the bioactive component that inhibits growth, repair, or healing of the target tissue is PDGF and/or VEGF.

Clause 5. The method of clause 1, wherein the target tissue is muscle, and wherein the bioactive component that inhibits growth, repair, or healing of the target tissue is PDGF and/or TGF-β1.

Clause 6. The method of clause 1, wherein the target tissue is bone, and wherein the bioactive component that inhibits growth, repair, or healing of the target tissue is noggin.

Clause 7. The method of any one of clauses 1-6, wherein the bioactive component that inhibits growth, repair, or healing of the target tissue is removed by affinity binding to a binding reagent, such as an antibody or antibody fragment.

Clause 8. The method of clause 7, wherein the binding reagent is bound to a bead or substrate, such as magnetic bead or a column medium.

Clause 9. The method of any one of clauses 1-8, wherein only, or essentially only, the deleterious factor is removed from the PRP.

Clause 10. The method of any one of clauses 1-9, wherein prior to removal of the deleterious factor, the platelets of the PRP are activated to produce a fibrin clot, and the fibrin clot is separated from the releasate from which the deleterious factor is removed.

Clause 11. The method of clause 10, wherein the platelets are activated using calcium, e.g., CaCl₂, and thrombin.

Clause 12. A method of growth, repair, or healing of a target tissue, in a patient, comprising contacting the target tissue with a platelet-rich-plasma-derived (PRP-derived) composition that is prepared by removal from activated PRP releasate a deleterious factor of the PRP comprising a bioactive component that inhibits growth, repair, or healing of the target tissue, such that as compared to PRP, the PRP-derived composition better promotes growth, repair, or healing of the tissue.

Clause 13. The method of clause 12, wherein the bioactive component that inhibits growth, repair, or healing of the target tissue is a cytokine or growth factor.

Clause 14. The method of clause 12, wherein the bioactive component that inhibits growth, repair, or healing of the target tissue has an immunomodulatory effect.

Clause 15. The method of clause 12, wherein the target tissue is cartilage, and wherein the bioactive component that inhibits growth, repair, or healing of the target tissue is PDGF and/or VEGF.

Clause 16. The method of clause 12, wherein the target tissue is muscle, and wherein the bioactive component that inhibits growth, repair, or healing of the target tissue is PDGF and/or TGF-β1.

Clause 17. The method of clause 12, wherein the target tissue is bone, and wherein the bioactive component that inhibits growth, repair, or healing of the target tissue is noggin.

Clause 18. The method of any one of clauses 12-17, wherein only, or essentially only, the deleterious factor is removed from the PRP.

Clause 19. The method of any one of clauses 12-18, wherein prior to removal of the deleterious factor, the platelets of the PRP are activated to produce a fibrin clot, and the fibrin clot is separated from the releasate from which the deleterious factor is removed.

Clause 20. The method of clause 19, wherein the platelets are activated using calcium, e.g., CaCl₂, and thrombin.

Clause 21. A platelet-rich-plasma-derived (PRP-derived) composition that is prepared by removal from activated PRP releasate a deleterious factor of the PRP comprising a bioactive component that inhibits growth, repair, or healing of a target tissue.

Clause 22. The composition of clause 21, wherein the bioactive component that inhibits growth, repair, or healing of the target tissue is a cytokine or growth factor.

Clause 23. The composition of clause 21, wherein the bioactive component that inhibits growth, repair, or healing of the target tissue has an immunomodulatory effect.

Clause 24. The composition of clause 21, wherein the bioactive component that inhibits growth, repair, or healing of the target tissue is PDGF and/or VEGF.

Clause 25. The composition of clause 21, wherein the bioactive component that inhibits growth, repair, or healing of the target tissue is PDGF and/or TGF-β1.

Clause 26. The composition of clause 21, wherein the bioactive component that inhibits growth, repair, or healing of the target tissue is noggin.

Clause 27. The composition of any one of clauses 21-26, wherein the bioactive component that inhibits growth, repair, or healing of the target tissue is reduced by at least 50%, 60%, 70%, 75%, 80%, 90%, 95%, 97.5%, 98%, 99%, or 99.9%, or any increment therebetween as compared to the composition prior to removal of the bioactive component.

Clause 28. The composition of any one of clauses 21-27, wherein only, or essentially only, the deleterious factor is removed from the PRP.

Clause 29. The composition of any one of clauses 21-28, wherein the PRP-derived composition is activated and is clot-free, such as where prior to removal or inactivation of the deleterious factor, the platelets of the PRP are activated to produce a fibrin clot, and the fibrin clot is separated from the releasate from which the deleterious factor is removed.

Clause 30. A kit comprising a container and, in the container, the composition of any one of clauses 21-29.

Clause 31. A kit comprising: a blood collection tube comprising an anticoagulant; a PRP activation reagent in a vessel; and an affinity-purification reagent comprising a binding reagent specific to, and for binding, a deleterious factor of activated PRP comprising a bioactive component that inhibits growth, repair, or healing of a target tissue, wherein the bioactive component optionally is VEGF, PDGF, TGF-β1, both VEGF and PDGF, both PDGF and TGF-β-1, or noggin.

### BRIEF DESCRIPTION OF THE DRAWINGS

FIGS. 1A-1C. TGF-β1(−) PRP injection showed improved muscle regeneration (H&E (original in color) in FIG. 1A and as quantified in FIG. 1B) and decreased collagen deposition (Picrosirus dark gray in FIG. 1A and as quantified in FIG. 1C) on a cardiotoxin induced muscle injury. *P<0.05 compared with control group, *P<0.05 compared with PRP group.

FIGS. 2A and 2B provide photomicrographs (FIG. 2A, Dystrophin, DAPI, and CD31 are green, blue, and red in original color image) and a graph (FIG. 2B) showing that TGF-β1(−) PRP injection increases the number of newly formed micro-vessels. *P<0.05 compared with control group.

FIGS. 3A and 3B provide photomicrographs (FIG. 3A, Dystrophin, DAPI, and PAX-7 are green, blue, and red in original color image) and a graph (FIG. 3B) showing TGF-β1(−) PRP injection increases the number of satellite cells. *P<0.05 compared with control group.

FIGS. 4A and 4B provide photomicrographs (FIG. 4A, CD68 and Transglutaminase-2 are green and red in original color image) and a graph (FIG. 4B) showing TGF-β1(−) PRP injection recruits more M2 macrophages. *P<0.05 compared with control group.

FIG. 5. is a graph showing sFLT-1 gene delivery significantly improves PRP effect on cartilage regeneration and repair. The effects of PRP and the combination therapy of PRP and sFlt-1 gene delivery via MDSC (M) were evaluated macroscopically and histologically for cartilage regeneration.

FIG. 6 is a graph showing the role of PDGF-AB in PRP on the proliferation of human blood vessel stem cells (myo-endothelial cells). Neutralizing antibody against PDGF-AB was added to the medium to block the function of PDGF-AB within PRP. *P<0.05. Data showed that PDGF-AB within PRP play a significant role for the stimulation of proliferation of myo-endothelial cells.

FIG. 7 is a graph showing quantitative analysis of Noggin in human PRP (n=6) by ELISA. The level of Noggin was found 1.8 times (1.67±0.322 ng/ml) higher than in Platelet Poor Plasma (PPP).

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases.

As used herein, the terms “comprising,” “comprise”, or “comprised,” and variations thereof, are meant to be open ended. The terms “a” and “an” are intended to refer to one or more.

As used herein, the term “patient” or “subject” refers to members of the animal kingdom including but not limited to human beings.

“Platelet-rich plasma” or “PRP” in its broadest sense is blood plasma with an enriched platelet content, where “enriched” is in reference to normal blood of a patient. Typically platelet content is enriched at least two-fold, and often at least five-fold or ten-fold. Platelet-rich plasma is typically prepared by centrifugation of anti-coagulase-treated blood obtained from one or more patients, and can be autologous. As an example, four forms of PRP are commonly-available: Pure Platelet-Rich Plasma (P-PRP) or leucocyte-poor PRP products are preparations without leucocytes and with a low-density fibrin network after activation; Leucocyte-PRP (L-PRP) products are preparations with leucocytes and with a low-density fibrin network after activation. This is the most common commercial PRP product; Pure platelet-rich fibrin (P-PRF) or leucocyte-poor platelet-rich fibrin preparations are without leucocytes and with a high-density fibrin network; and Leucocyte- and platelet-rich fibrin (L-PRF) or second-generation PRP products are preparations with leucocytes and with a high-density fibrin network (Dhurat et al. Principles and Methods of Preparation of Platelet-Rich Plasma: A Review and Author's Perspective. Journal of Cutaneous and Aesthetic Surgery. 2014; 7(4):189-197).

For use in preparing the PRP composition as described herein, a number of methods are broadly-known for preparation of PRP. The PRP may comprise leukocytes, or may have leukocytes removed, depending on the preparation method. In one embodiment, leukocytes are removed during PRP preparation. In another embodiment, leukocytes are not removed from the PRP. In one non-limiting example, blood is collected in tubes containing anticoagulants. A platelet layer, a buffy coat layer, and a red blood cell (RBC) layer are produced. For production of leukocyte-poor PRP, the platelet layer and only the superficial buffy coat layer are transferred to a clean tube. For preparation of leukocyte-containing PRP, the platelet layer and buffy coat layer are transferred to a clean tube. The second tube is spun in a centrifuge resulting in a soft platelet pellet at the bottom of the tube. A portion, e.g., ⅔, of the platelet-poor top volume is removed, and the platelet pellet is then dispersed, e.g., homogenized, in the remaining plasma. Other methods may be used to concentrate and re-suspend platelets and other blood fractions to provide the PRP starting material as described herein.

PRP contains concentrated growth factors, potentially at optimal ratios, that could accelerate tissue healing, such as platelet derived growth factor (PDGF), vascular endothelial growth factor (VEGF), insulin-like growth factor (IGF), transforming growth factor-beta (TGF-beta), epithelial growth factors (EGF), etc. However, PRP also contains high amounts of “negative/deleterious” cytokines or growth factors such as inflammatory cytokines, microparticles, reactive oxygen species (ROS), and matrix metalloproteinase (MMPs) which could result in exacerbated or increased tissue damage. Although most the current work being performed on PRP attempts to identify various growth factors and cytokines present within PRP that are responsible for tissue healing, which is a very difficult task to achieve, we developed a novel approach where we will customize the PRP to be tissue-specific by eliminating “negative/deleterious” factors for any specific tissue to further improve the beneficial effects of PRP.

Platelet activation refers to the degranulation of platelets in PRP, releasing the factors contained in the PRP, as well as causing the formation of a clot. A number of methods may be used to activate platelets in PRP, including, for example and without limitation, adding calcium (e.g., CaCl₂)), adding thrombin (e.g., autologous thrombin), adding both calcium and thrombin, or collagen, type I. In embodiments of the methods and compositions described herein, deleterious factors are removed from PRP that has been activated, producing a clot and a releasate. The clot is typically separated from the releasate and is removed from the releasate to facilitate further processing as described herein. The clot can typically be removed by centrifugation, leaving a supernatant releasate from which one or more deleterious factor(s) can be removed, for example by affinity purification.

According to one aspect of the present invention, a method of preparing a platelet-rich-plasma-derived (PRP-derived) composition is provided. The composition may be used in regeneration of a tissue, such as repair of injured or damaged tissue, or repair of a congenital defect in the tissue. Production or regeneration of any specific tissue may be promoted by certain pro-regenerative substances, factors, or components (used interchangeably herein), many of which are present in PRP. Likewise, production or regeneration of any specific tissue may be hampered by certain bioactive substances, factors, or components, many of which are present in PRP. The method therefore can include removing from PRP, e.g., from releasate produced from activated leukocyte-containing PRP, leukocyte-poor PRP, or leukocyte-free PRP, a bioactive component of PRP that hampers or inhibits desirable regeneration, growth, repair, or healing of the tissue, referred to herein as a “negative/deleterious factor”, or simply a “deleterious factor” of the PRP, such that as compared to PRP, the PRP-derived composition better promotes growth, repair, or healing of the target tissue (the tissue to be regenerated, repaired, grown, etc.), for example, as indicated by better, e.g., faster, cell or tissue growth, or lack of undesirable consequences, such as pro-inflammatory effect, scarring/collagen deposition, or abnormal tissue structure.

Platelets, as a main component of the PRP, contain more than 1100 different proteins, with numerous post-translational modifications, resulting in over 1500 protein-based bioactive factors (see, e.g., Pavlovic V, el al. Platelet Rich Plasma: a short overview of certain bioactive components. Open Med (Wars). 2016; 11(1):242-247. Published 2016 Aug. 12). As such, by “substances”, “factors”, “components” or “bioactive component(s)” of PRP, it is meant any compound, substance, factor, or component present in PRP and/or platelets, such as proteins or polypeptides, including, without limitation, adhesive proteins, growth factors, cytokines, chemokines, angiogenic factors, clotting factors, clotting inhibitors, integral membrane proteins, and immune mediators. Reference herein to “deleterious factors”, or “negative/deleterious factors” refers to factors that are deleterious, or otherwise negatively impact growth, of any specific tissue, such as cartilage, muscle, or bone, and therefore the deleterious factors are referenced in the context of a specific tissue, though they may be deleterious to multiple tissue types, or even to all tissue types.

For instance, angiogenic growth factors, such as VEGF and PDGF, are capable of promoting blood vessel invasion (angiogenesis). Angiogenesis is known to compromise cartilage regeneration. As such, as supported by the Examples below, customized PRP from which angiogenic factors, such as, for example and without limitation, VEGF and/or PDGF, are removed, is expected to be beneficial for articular cartilage healing.

Similarly, TGF-β1 and PDGF induces muscle fibrosis during muscle healing, which is known to compromise the healing process. As such, and as supported by the examples below, customized PRP (TGF-β1 and/or PDGF minus) is beneficial for muscle healing.

As a further example, elevated levels of the well-known bone morphogenetic protein (BMP) antagonist noggin within the PRP would be expected to impair osteogenesis. Eliminating noggin from PRP is therefore expected to improve the ability of PRP to promote bone regeneration and repair.

A “binding reagent” is a reagent, compound, or composition, e.g., a ligand, able to specifically bind a target compound, such as, for example and without limitation: TGF-β1, PDGF, noggin, or VEGF. Binding reagents include, without limitation, antibodies (polyclonal, monoclonal, humanized, etc.), antibody fragments (e.g., a recombinant scFv), antibody mimetics such as affibodies, affilins, affimers, affitins, alphabodies, anticalins, avimers, DARPins, fynomers, monobodies, nucleic acid ligands (e.g., aptamers), engineered proteins, antigens, epitopes, haptens, or any target-specific binding reagent. In aspects, binding reagents includes as a class: monoclonal antibodies, or derivatives or analogs thereof, including without limitation: Fv fragments, single chain Fv (scFv) fragments, Fab′ fragments, F(ab′)₂ fragments, single domain antibodies, camelized antibodies and antibody fragments, humanized antibodies and antibody fragments, multivalent versions of the foregoing, and any paratope-containing compound or composition; multivalent activators including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (e.g., leucine zipper or helix stabilized) scFv fragments; nucleic acids and analogs thereof that bind a target compound; or receptor molecules which naturally interact with a desired target molecule. Antibodies and other binding reagents that specifically bind their target compounds, such as TGF-β1, PDGF, noggin, or VEGF, are generally commercially-available, or can be generated by a person of ordinary skill in the art using common methods. For example, various antibodies and antibody fragments, specific to TGF-β1, PDGF, noggin, or VEGF, are broadly-available from commercial sources, such as ThermoFisher Scientific/Invitrogen (US) or AbCam (UK), among many other sources.

For affinity purification methods, according to common practice, a binding reagent specific to the deleterious factor may be conjugated (covalently linked) or otherwise attached to a surface, such as beads, e.g., magnetic beads or agarose beads (e.g., SEPHAROSE beads), and the PRP may be contacted with the conjugated binding reagent to bind the deleterious factor, and the PRP is then separated from the PRP, thereby producing tissue-customized PRP in which the deleterious factor is removed. Once the bound deleterious factor is removed from the PRP, the surface-conjugated binding reagent can be reconstituted by dissociating the deleterious factor from the binding reagent by washing according to common practice. The binding reagent may be a monoclonal or polyclonal antibody, an scFv, or any other suitable binding reagent. The surface may be a magnetic bead, and the bound deleterious factor may then be removed by magnetic removal of the magnetic beads from the PRP. The surface may be particles or beads retained in a column. A person of ordinary skill can readily envision a large variety of ways affinity purification may be used to remove deleterious factors from the PRP.

In practice, to remove factors from PRP, the PRP may first be activated, e.g., with calcium and thrombin, centrifuged to remove the clot, and then the deleterious factor(s) may be removed, e.g., by affinity by binding to one or more binding reagents specific to the deleterious factor(s), for example as described herein.

The deleterious agent or bioactive component that inhibits growth, repair, or healing of the target tissue may be reduced by at least 50%, by at least 60%, by at least 70%, by at least 75%, by at least 80%, by at least 90%, by at least 95%, by at least 97.5%, by at least 98%, by at least 99%, or by at least 99.9%, or any increment therebetween, as compared to the composition prior to removal of the deleterious agent or bioactive component. “Reduced” may refer to mass, molar percent, percent activity, or any other useful measure of activity, such as a percent reduction in the number of detectable molecules, a percent reduction of active molecules, or a percentage reduction of mass.

The compositions described herein, that is PRP, e.g., PRP releasate, in which one or more deleterious agents is removed, may be packaged in a kit. A kit minimally comprises a vessel, such as a tube, flask, pouch, medical syringe, reservoir, cartridge, or the like, containing the composition according to any aspect, embodiment, or example provided herein. The vessel may be watertight or hermetically-sealed. The vessel may be contained within an additional container, such as a mylar pouch, box, or shipping container, as is commonly used in the medical arts. The kit may also include a medical syringe and a needle, catheter, nozzle, tube, or other applicator for depositing the composition at a site in a patient. Alternatively, where the composition is not provided, the kit may comprise a venipuncture apparatus, such as a needle and associated tubes and connectors, as are known in the phlebotomy arts. The kit may comprise vessels (e.g., tubes) for collecting and processing a patient's blood into PRP and optionally the final processed PRP product in which a deleterious agent is removed. The kit may comprise a vacuum blood collection tube optionally containing an anticoagulant, such as heparin. Platelet activator, such as calcium and thrombin, may be included in the kit, as well as additional tube(s) for use in processing the PRP. Affinity-purification reagents may be included in the kit to remove the deleterious factor(s) from the PRP, e.g., from releasate from activated PRP. Antibody-conjugated magnetic beads or affinity columns or devices may be included in the kit.

The examples below show that PRP can be used beneficially in the growth and repair of specific tissues if negative/deleterious factors are removed from the PRP, e.g., PRP releasate, before use of the PRP. Tissue-customized PRP can therefore be used for specific musculoskeletal tissue repair by eliminating targeted factors from autologous or allogenic PRP. The targeted factors may be eliminated by affinity purification, such as via neutralization antibodies, which can be conjugated to a suitable surface or support such as magnetic beads, or in a column, as are broadly-known. This technique are easily added to current clinical PRP isolation protocols to provide tissue specific PRP products. In one example, the PRP is PRP releasate. As above, PRP releasate is a PRP fraction prepared by activation of the PRP to form a clot (fibrin network), e.g., with calcium and thrombin, and, typically removing the clot, e.g., by centrifugation where the clot is pelleted and the PRP releasate is the supernatant. Affinity purification may then be performed on the PRP releasate to remove any deleterious factor(s).

### Example 1. Customization of PRP on Muscle Healing

Rationale: The rationale for supporting the clinical use of PRP is that abundant autologous growth factors, such as vascular endothelial growth factor (VEGF), insulin-like growth factor (IGF), and fibroblast growth factor (FGF), etc., released by platelets accelerate muscle cell proliferation and differentiation and promote muscle regeneration. However, PRP also contains high levels of TGF-β1 and PDGF that are key factors responsible for the development of fibrosis. Concerns remain that PRP injection may lead to elevated fibrosis, and therefore hinder optimal muscle healing. We propose that TGF-β1-minus and/or PDGF-minus PRP administration will result in improved skeletal muscle healing than regular PRP through the reduction of fibrosis, increased activation of satellite cells, and modulation of macrophage polarization.

Preliminary data: A neutralizing Ab against TGF-β1 was utilized by our group with the idea of blocking the function of TGF-β1 within PRP while keeping the functions of beneficial factors for more optimized muscle recovery.

In brief, rat PRP was prepared by centrifugation, activated by freeze-thaw cycles, and PRP releasate was collected and mixed with TGF-β1 neutralization Abs. The TGF-β1 neutralized PRP was injected to the muscle injury sites and its effects were compared with original PRP and saline controls.

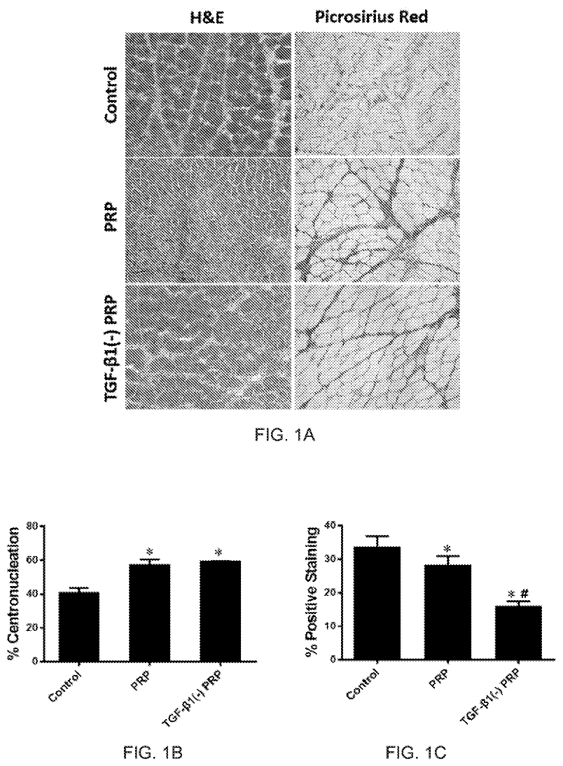

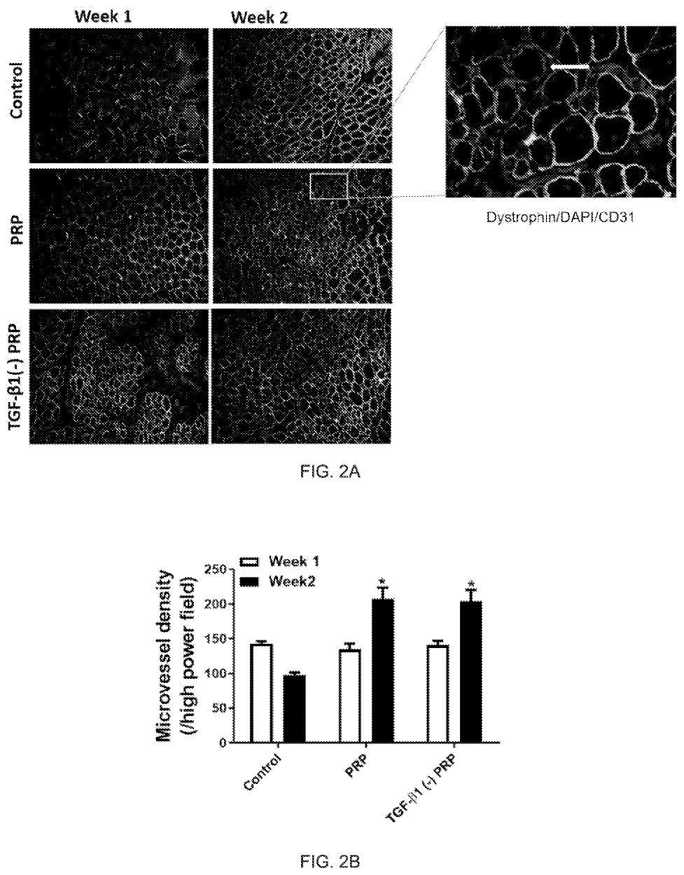

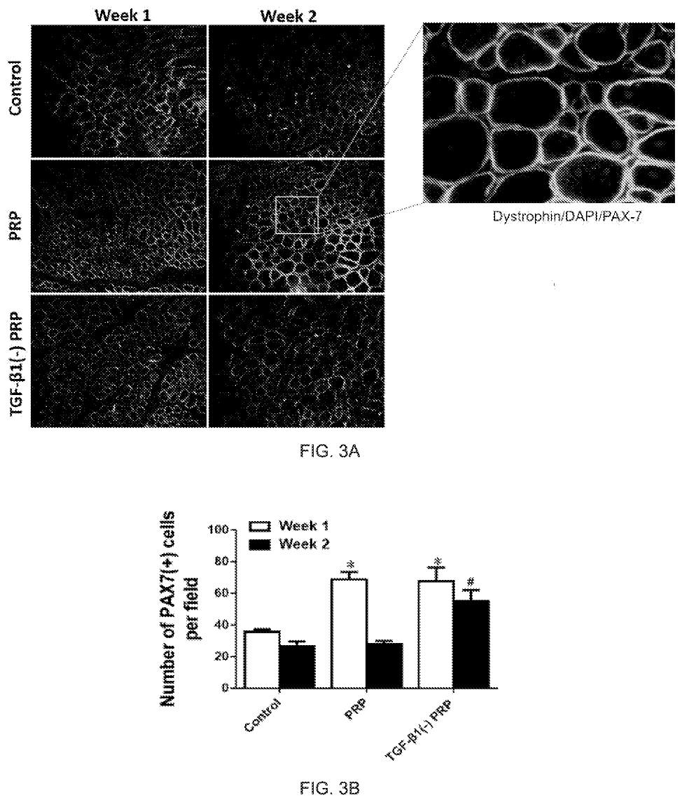

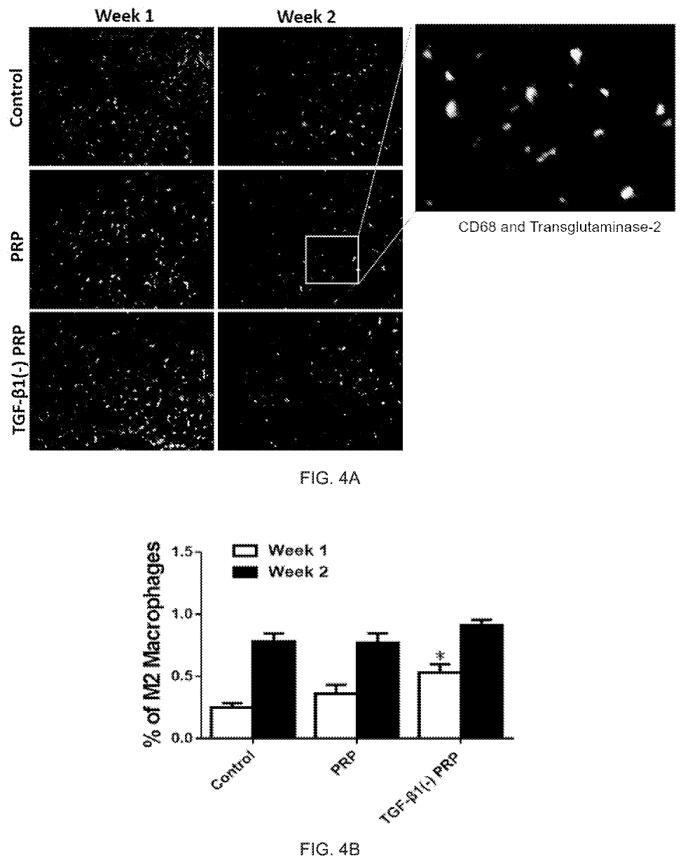

Our exciting data demonstrated that neutralizing TGF-β1 within PRP significantly promotes muscle regeneration (FIG. 1A H&E and FIG. 1B) while significantly decreasing collagen deposition (FIG. 1A Picrosirus Red and FIG. 1C) in a cardiotoxin induced skeletal muscle injury model (Li, H., et al., Customized platelet-rich plasma with transforming growth factor beta1 neutralization antibody to reduce fibrosis in skeletal muscle. Biomaterials, 2016. 87: p. 147-56). Not only did the neutralization reduce fibrosis, it enhanced angiogenesis (FIGS. 2A and 2B), prolonged satellite cell activation (FIG. 3), and recruited a great number of M2 macrophages to the injury site (FIGS. 4A and 4B), which also contributed to the efficacy that the TGF-β1 minus PRP had on muscle healing.

Besides TGF-β1, PDGF which is also abundant in PRP, has been shown to mediate muscle fibrosis during muscle disease and after injury. Elevated PDGF expression is observed post-wounding and causes neutrophils, macrophages, fibroblasts, and smooth muscle cells to proliferate and migrate into the wound site. Injection of neutralizing PDGF-alpha receptor-specific antibody attenuated atrial fibrosis (Liao, C. H., et al., Cardiac mast cells cause atrial fibrillation through PDGF-A-mediated fibrosis in pressure-overloaded mouse hearts. J Clin Invest, 2010. 120(1): p. 242-53). Furthermore, isoforms of PDGF and its receptors were found significantly overexpressed in the skeletal muscle of muscle dystrophy patients, suggesting that PDGF and its receptors are significantly involved in the active stage of muscle destruction and are associated with the initiation or promotion of muscle fibrosis. Taken together, TGF-β1 and PDGF are both pro-fibrotic factors in PRP and neutralizing their activities may potentiate the beneficial effects of PRP on muscle healing via reducing fibrosis.

### Example 2. Customization of PRP on Cartilage Regeneration

Rationale: Recently, the usage of PRP to improve AC healing has been getting more attention. Initial experiments in the laboratory have shown the anabolic effects of PRP on cartilage repair by showing that PRP stimulates the proliferation and matrix biosynthesis of chondrocytes. The use of PRP has also been proposed to treat osteoarthritis (OA) and rheumatoid arthritis (RA) because of its ability to inhibit inflammation; however, the evidence to support PRPs use for clinical treatment is still controversial. Moreover, It is well known that AC is mainly avascular and that endogenous inhibitors of angiogenesis in cartilage tissue, such as Troponin I (TnI) and Chondromodulin-1 (chM-1), may play an important role in the resistance of cartilage angiogenesis which consequently prevented endochondral bone formation and maintained the cartilage's hyaline-like phenotype. Furthermore, angiogenesis or neovascularization has been shown to play a key role in the pathogenesis of OA. Angiogenic growth factors lead to chondrocytes that have a hypertrophic phenotype, and the penetration of blood vessels, originating from subchondral bone into the cartilage, which promotes the subsequent outgrowth of bone and sensory neurons that represents a major cause of pain. Anti-angiogenesis therapy has been proven effective at preventing these deleterious effects in some studies. Although PRP is rich in some growth factors, such as TGF-β, which have beneficial effects on cartilage, other angiogenic growth factors, such as VEGF and PDGF might be harmful for cartilage regeneration. Indeed, previous studies have shown that blocking VEGF, using soluble fms-like tyrosine kinase-1 (sFlt-1), improved the chondrogenic potential of muscle derived stem cells (MDSCs) and further improved the repair of AC after OA in vivo. Blocking angiogenesis is therefore expected to be an effective approach to improve AC healing after injury. We propose that VEGF-minus and/or PDGF-minus PRP administration will result in improved articular cartilage regeneration than regular PRP through the reduction of angiogenesis and maintain the hyaline cartilage phenotype.

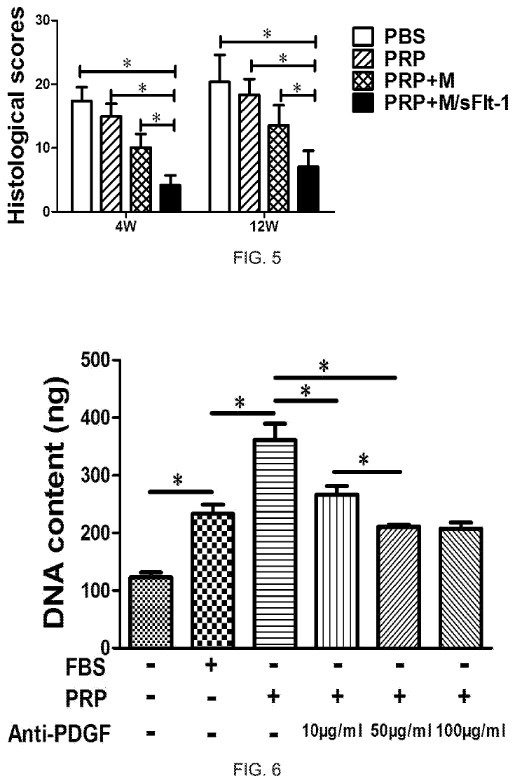

Our preliminary data on a Mono-iodoacetate (MIA)-induced rat OA model showed that blocking VEGF through gene delivery of sFlt-1 significantly improved the beneficial effect of PRP on AC repair (FIG. 5). These data indicate that VEGF in PRP may not be beneficial for the cartilage repair; thus, eliminating VEGF within PRP may improve the therapeutic effects of PRP for cartilage regeneration and repair. We will perform customization of PRP through the elimination of VEGF via neutralization Abs or other technique, instead of gene transfer, and determine whether a significant improvement of cartilage healing through a reduction of angiogenesis will be observed, when compared to non-customized PRP.

In addition, another major angiogenic growth factor in the PRP, PDGF-AB (117.5±63.4 ng/ml), will be also be eliminated and tested. We have found that the proliferative effect that PRP has on muscle stem cells is eliminated when PDGF-AB is neutralized indicating that PDGF-AB is a potential active substance that will promote the proliferation of blood vessel stem cells and potentially angiogenesis (FIG. 6). We can also eliminate both VEGF and PDGF-AB to test if significant beneficial effects could be observed in the AC repair process.

### Example 3. Customization of PRP on Bone Healing

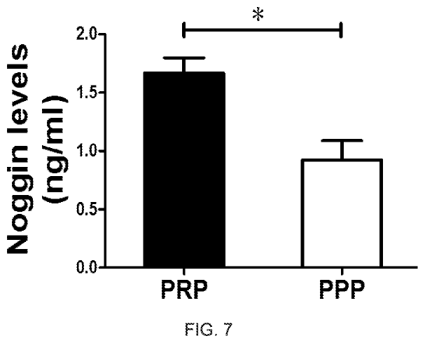

Rationale and preliminary data: PRP contains high concentrations of angiogenic growth factors, and members of the TGF-3 super family, such as TGF-β1 and the bone morphogenetic proteins (BMPs), which contribute to the angiogenesis, tissue repair and the process of bone mineralization. As a result, PRP has been proposed to be a potential therapeutic material to promote bone healing; however, the efficacy of PRP to accelerate bone healing continues to be controversial. PRP not only contains the aforementioned growth factors that could contribute to bone healing, but we have also observed that PRP contains high levels of noggin (FIG. 7), which is a strong antagonist of the BMPs and could reduce bone formation. Removing noggin within the PRP may improve the bone healing ability of PRP. We propose to customize PRP by eliminating noggin and determine whether a significant improvement of bone healing will be observed when compared to the non-customized PRP.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A method of preparing a platelet-rich-plasma-derived (PRP-derived) composition for use in regeneration of a target tissue, comprising: removing from activated PRP releasate a deleterious factor of the PRP comprising a bioactive component that inhibits growth, repair, or healing of the target tissue.

2. The method of claim 1, wherein the bioactive component that inhibits growth, repair, or healing of the target tissue is a cytokine or growth factor and/or has an immunomodulatory effect.

3. (canceled)

4. The method of claim 1, wherein the target tissue is cartilage, and wherein the bioactive component that inhibits growth, repair, or healing of the target tissue is PDGF and/or VEGF.

5. The method of claim 1, wherein the target tissue is muscle, and wherein the bioactive component that inhibits growth, repair, or healing of the target tissue is PDGF and/or TGF-β1.

6. The method of claim 1, wherein the target tissue is bone, and wherein the bioactive component that inhibits growth, repair, or healing of the target tissue is noggin.

7. The method of as, wherein the bioactive component that inhibits growth, repair, or healing of the target tissue is removed by affinity binding to a binding reagent, such as an antibody or antibody fragment.

8. The method of claim 7, wherein the binding reagent is bound to a bead or substrate, such as magnetic bead or a column medium.

9. The method of claim 1, wherein only, or essentially only, the deleterious factor is removed from the PRP.

10. The method of claim 1, wherein prior to removal of the deleterious factor, the platelets of the PRP are activated to produce a fibrin clot, and the fibrin clot is separated from the releasate from which the deleterious factor is removed.

11. (canceled)

12. A method of growth, repair, or healing of a target tissue, in a patient, comprising contacting the target tissue with a platelet-rich-plasma-derived (PRP-derived) composition that is prepared by removal from activated PRP releasate a deleterious factor of the PRP comprising a bioactive component that inhibits growth, repair, or healing of the target tissue, such that as compared to PRP, the PRP-derived composition better promotes growth, repair, or healing of the tissue.

13. The method of claim 12, wherein the bioactive component that inhibits growth, repair, or healing of the target tissue is a cytokine or growth factor and/or has an immunomodulatory effect.

14. (canceled)

15. The method of claim 12, wherein the target tissue is cartilage, and wherein the bioactive component that inhibits growth, repair, or healing of the target tissue is PDGF and/or VEGF.

16. The method of claim 12, wherein the target tissue is muscle, and wherein the bioactive component that inhibits growth, repair, or healing of the target tissue is PDGF and/or TGF-β1.

17. The method of claim 12, wherein the target tissue is bone, and wherein the bioactive component that inhibits growth, repair, or healing of the target tissue is noggin.

18. The method of claim 12, wherein only, or essentially only, the deleterious factor is removed from the PRP.

19. The method of claim 12, wherein prior to removal of the deleterious factor, the platelets of the PRP are activated to produce a fibrin clot, and the fibrin clot is separated from the releasate from which the deleterious factor is removed.

20. (canceled)

21. A platelet-rich-plasma-derived (PRP-derived) composition that is prepared by removal from activated PRP releasate a deleterious factor of the PRP comprising a bioactive component that inhibits growth, repair, or healing of a target tissue.

22. (canceled)

23. (canceled)

24. The composition of claim 21, wherein the bioactive component that inhibits growth, repair, or healing of the target tissue is PDGF, VEGF, TGF-β1, and/or noggin.

25. (canceled)

26. (canceled)

27. (canceled)

28. The composition of claim 21, wherein only, or essentially only, the deleterious factor is removed from the PRP.

29. (canceled)

30. A kit comprising a container and, in the container, the composition of claim 21.

31. (canceled)
